(19) United States
(12) Patent Application Publication
(10) Pub. No.: US 2007/0106125 A1
(43) Pub. Date: May 10, 2007

(54) Biosensor capable of setting standard measuring values

(75) Inventors: Paul Yang, Taipei (TW); Ya-Hsin Chuang, Taipei (TW); Shih-Chieh Hsiao, Taipei (TW); Hsin-Yi Lin, Taitung (TW)

Correspondence Address: BACON & THOMAS, PLLC, 625 SLATERS LANE, FOURTH FLOOR, ALEXANDRIA, VA, 22314, US

(73) Assignee: HEALTH & LIFE CO., LTD, Taipei (TW)

(21) Appl. No.: 11/230,485

(22) Filed: Sep. 21, 2005

(51) Int. Cl. A61B 5/00

(52) U.S. Cl. 600/300; 128/923

(57) ABSTRACT

A biosensor capable of setting standard measuring values includes a detecting unit coupled with an analog-to-digital converting unit, and the analog-to-digital converting unit is coupled to a processor, and the processor is coupled separately with a setting unit, a display unit, a data storage unit, a reminding unit, and a power supply unit, such that when the detecting unit examines biological testing body, the analog-to-digital converting unit converts test information into a digital data. In the meantime, the processor retrieves the standard values prestored in the data storage unit for a comparison. After the data is processed, the data is displayed by the display unit and the reminding unit. In the meantime, users can use the setting unit to change the default settings of the standard values. The reminding unit will use different lights or sounds to remind users, so that users need not to memorize the meanings of the complicated measured values, but can immediately know about the normal or abnormal signals directly by different lights or sounds.

### BACKGROUND OF THE INVENTION

1. Field of the Invention

The present invention relates to a biosensor capable of setting standard measuring values, and more particularly to a biosensor that allows users to set the standard measuring values again by themselves according to a new health reference standard amended by the World Health Organization (WHO) or the American Diabetes Association (ADA) or related medical organizations and the change of standard measuring values for the patient's anamnesis and the recommendations made by medical staffs. With different light signals or sounds of the biosensor, users do not have to memorize the complicated measuring values in order to understand the normal or abnormal conditions of the patient, but the users can simply and directly learn them from the light signals or sounds.

2. Description of the Related Art

Since the concept of household nursing becomes increasingly popular, and there are various different kinds of sensor products for a fast, cheap, non-professional operation, and these sensor products include a blood sugar meter, an electronic ear thermometer, and an electronic sphygmomanometer and allow users to understand their health conditions without wasting much time or money to make a special trip to a hospital or clinic and obtain assistance from medical people for taking the test. Users simply need to have their own medical measuring instrument, so that they can know their body conditions anywhere and anytime, so as to avoid possible delays of a patient's conditions.

However, general users are not medically trained and usually do not understand the real meaning of the values measured by various different medical instruments. Although some medical instrument manufacturers pre-store the health standard values or dangerous values in the instrument according to the standards established by WHO or ADA and use different methods to remind users to pay attention to the normal or dangerous values by means of different color blinking lights or warning sounds, users still have to know and get used to the normal and abnormal values amended by WHO or ADA due to the advancement of medical science and the accumulation of clinical experience. Particularly, manufacturers usually remind users by a factory default setting instead of providing users a way of adjusting or changing the standard measuring values. Therefore, users may misjudge or delay a patient's conditions easily.

Each user may have standard values deviated from the published standard values according to the family inherent cause or illness condition. For example, a diabetic usually has a higher average blood sugar level than normal people. In this case, the dangerous reminding value should be increased to meet the average measuring condition. The general normal range of blood sugar level is approximately 60-115 mg/dl (3.4˜6.4 mmol/L) depending on the time of a person's last meal. The sugar blood level drops below 60 mg/dl (3.4 mmol/L) or even to 50 mg/d (2.8 mmol/L) when a person has an empty stomach, but there is nothing seriously wrong about such readings. This condition is common for healthy women, particularly after a long period of fasting. Although the reading actually meets the average condition, the dangerous reminding values preset by manufacturers cannot be adjusted accordingly and it may cause unnecessary panic to users.

In the meantime, the measured blood sugar level relates to the body mass index (BMI) of the patient. For those who have a BMI higher than a standard value, the measured sugar blood level of these people is usually higher. Information of this sort cannot be shown or reflected in the prior art device or provided to medical people in order to make correct judgments and decisions.

### SUMMARY OF THE INVENTION

In view of the shortcomings of the prior art, the inventor of the present invention aimed at the problem and tried to find a way of overcoming the shortcomings and conducted extensive researched and experiments to find a feasible solution, and finally invented a biosensor capable of setting standard measuring values in accordance with the present invention.

Therefore, it is a primary object of the present invention to provide a biosensor capable of setting standard measuring values comprising a setting unit, so that users can set the normal or abnormal values again by themselves according to the amendments made by related health organizations or the instructions given by medical people to reflect the actual conditions of each user.

It is another object of the invention to provide a biosensor capable of setting standard measuring values comprising a detecting unit coupled with an analog-to-digital converting unit, and the analog-to-digital converting unit is coupled to a processor, and the processor is coupled separately to a setting unit, a display unit, a storage unit, and a power supply unit, so that the detecting unit is used to detect a biological testing body, the measured value is converted into a digital data by the analog-to-digital converting unit. In the meantime, the preset standard values stored in the processor by the setting unit such as the normal standard values or dangerous values are retrieved by the storage unit for comparison, and a relative reminding method (such as using different lights to indicate normal and abnormal conditions) with respect to the default settings is used by a reminding unit to remind users about their conditions. Therefore, users can know about their body conditions in a simple and easy manner.

Another object of the invention is to provide a biosensor capable of setting standard measuring values comprising a storage unit for storing a body mass index (BMI) setup mode, so that when a user carries out a test, the setting unit inputs the values required by the body mass index (BMI) and the processor stores the values in a storage unit for accurately reflecting the actual conditions during the measuring process and allowing doctors to make correct judgments.

A further object of the invention is to provide a biosensor capable of setting standard measuring values comprising a display unit such as a liquid crystal display module for displaying the measured values and the result on a LCD display.

### BRIEF DESCRIPTION OF THE DRAWINGS

To make it easier for our examiner to understand the objective, shape, assembly, structure, characteristics and performance of the present invention, the following embodiments accompanied with the related drawings are described in details.

FIG. 1 is a perspective view of the present invention;

FIG. 2 is a block diagram of the units of the present invention; and

FIG. 3 is a flow chart of the operation of the present invention.

### DETAILED DESCRIPTION OF THE PREFERRED EMBODIMENTS

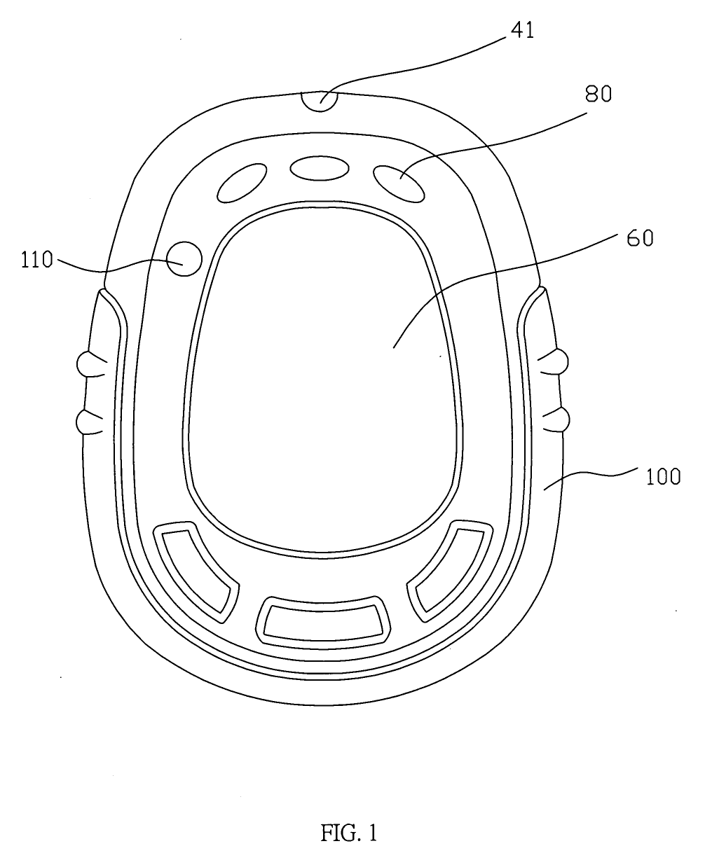

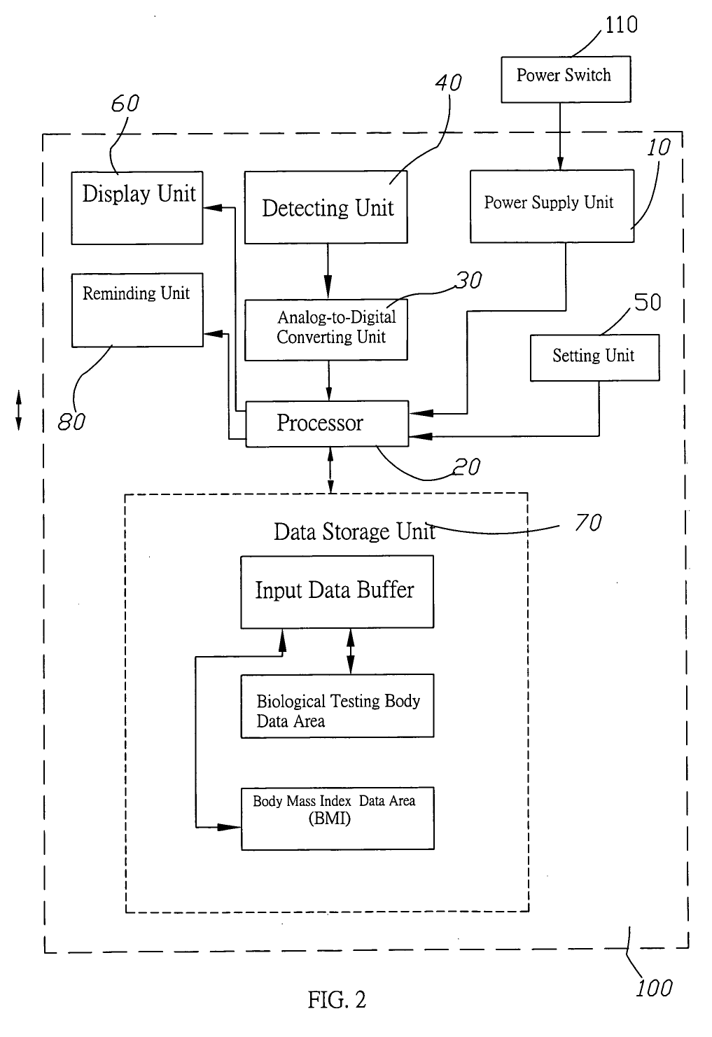

Referring to FIGS. 1 and 2, the present invention discloses a biosensor capable of setting standard measuring values that includes a casing 100, and the casing 100 could be in any shape, and the casing 100 has a power supply unit 10 thereon, and the power supply unit 10 is coupled to the power switch 110 on the casing 100, so that when the biosensor is in use, users can switch the power switch 110 to turn the power on or off.

The casing 100 includes a processor 20, and the processor 20 is coupled to the power supply unit 10, and the processor 20 is coupled to an analog-to-digital converting unit 30, and the analog-to-digital converting unit 30 is coupled to a detecting unit 40, and the detecting unit 40 includes a detecting opening 41 for inserting a strip of a testing body, and the analog-to-digital converting unit 30 converts a signal detected by the detecting unit 40 into a digital signal.

The processor 20 is coupled separately to a setting unit 50, a display unit 60, a storage unit 70 and a reminding unit 80, wherein the display unit 60 could be a liquid crystal display (LCD) module for displaying test results. The setting unit 50 could be a press button for changing the settings of various health standard values by users. Further, the data storage unit 70 could be a memory, and the data storage unit 70 is used for storing various different health standard values. Further, the reminding unit 80 could be a light emitting diode for displaying different colors for the testing results by the reminding unit 80. For example, the reading detected 2 hours before or after a meal is 80˜115 mg/dl (4.5˜6.4 mmol/L) indicated by a green light and representing a normal reading, and the reading below 80 mg/dl (4.5 mmol/L) in the range of 126˜200 mg/dl (7.0˜11.1 mmol/L) is indicted by a yellow light representing that attentions should be paid, and the reading below 60 mg/dl (3.44 mmol/L) or greater than 200 mg/dl (11.1 mmol/L) is indicated by a red light representing a serious abnormal problem.

Referring to FIGS. 1 to 3 for the use of the biosensor, the power is turned on first, so that the power supply unit 20 supplies electric power to the related connecting components. In the meantime, the strip with the testing body is inserted into the detecting opening 41, and the data detected by the unit is sent to the processor 20 through the analog-to-digital converting unit 30. If the processor 20 receives the data, the set values stored in the data storage unit 70 will be retrieved and compared with the data. If the data is less than the set value, then the result is displayed on the display unit 60, and the reminding unit 80 displays a green light. If the data exceeds the set value, then the processor 20 will generate a warning signal and the reminding unit 80 will display a preset warning signal (such as a yellow light or a red light), and the warning signal is stored into the data storage unit 70.

Then, the setting unit 50 selects an input mode. For example, the processor 20 will retrieve related data from the data storage unit 70 if the BMI is added, and then will request users to input the required values which are the height and weight of the users in this preferred embodiment. After the processor 20 processes the values, these values and the detected values are stored in the data storage unit 70. Therefore, obesity is often considered as one of the factors affecting the blood sugar level.

Due to WHO or ADA organization, or some patients having familial diabetic inheritance or inborn hypoglycemia, the average blood sugar value will be higher (lower) than the factory default settings of the standard values. Therefore, the settings can be set again by the setting unit 50 according to the health reference standard value announced by related health organizations or the instruction given by doctors. In the meantime, the recently set warning value is displayed by the reminding unit 80, and the values are stored together in the data storage unit 70. When a test is performed, the recently set standard values and the measured values are compared, and then displayed by the display unit 60, and the reminding unit 80 obtains the corresponding warning to record the measured sugar blood data for the reference of the doctors and allows the doctors to make decisions if it is necessary to change the medication of a patient.

In summation of the above description, the present invention herein enhances the performance and overcomes the shortcoming of the prior art, and further complies with the patent application requirements.

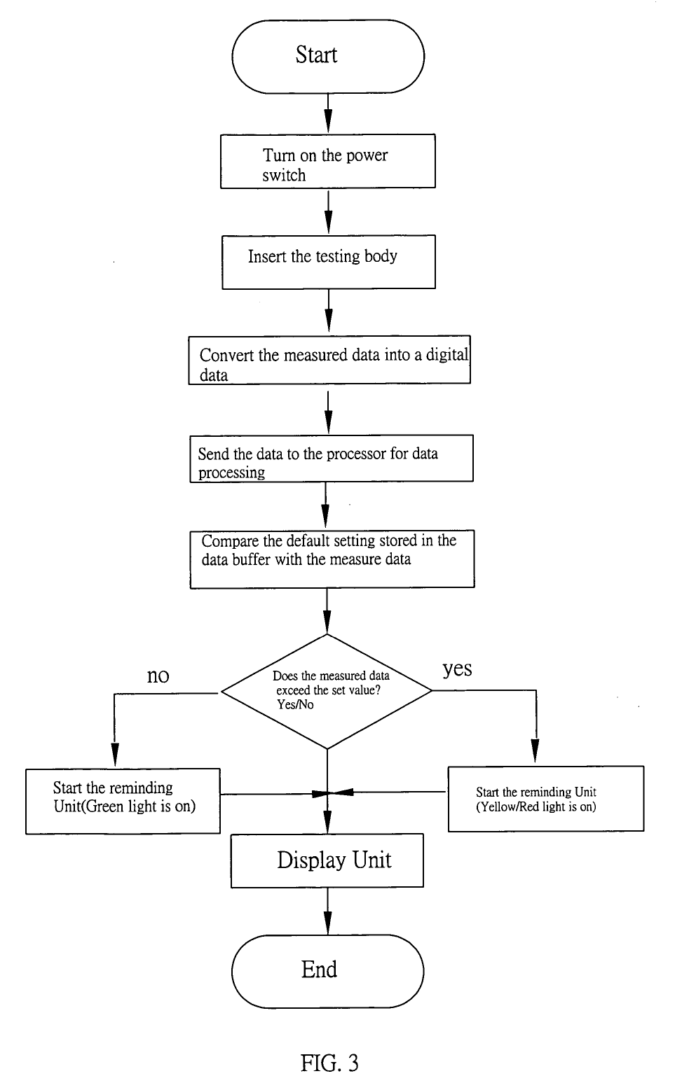

While the invention has been described by way of example and in terms of a preferred embodiment, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.
110 Power Switch
60 Display Unit
40 Detecting Unit
10 Power Supply Unit
80 Reminding Unit
30 Analog-to-Digital Converting Unit
50 Setting Unit
20 Processor
70 Data Storage Unit
   Input Data Buffer
   Biological Testing Body Data Area
   Body Mass Index (BMI) Data Area
FIG. 2
Start
Turn on the power switch.
Insert the testing body.
Convert the measured data into a digital data.
Send the data to the processor for data processing.
Compare the default setting stored in the data buffer with the measure data.
Does the measured data exceed the set value? Yes/No
Start the reminding Unit (Green light is on.)
Start the reminding Unit (Yellow/Red light is on.)
Display Unit
End
FIG. 3

What is claimed is:

1. A biosensor capable of setting standard measuring values, comprising:
a detecting unit, for detecting a testing body;
an analog-to-digital converting unit, coupled with said detecting unit for converting an analog signal of said detected biological testing body into a digital signal;
a processor, coupled with said analog-to-digital converting unit for determining and processing;
a display unit, coupled with said processor for displaying a test result;
a setting unit, coupled with said processor for changing the related setting of various health standard values;
a storage unit, coupled with said processor for storing various reference variables and measured results;
a reminding unit, coupled with said processor for reminding users about the comparison between the measured values of said testing body and said standard values;
a power supply unit, coupled with said detecting unit, said processor, said display unit, and said setting unit for supplying the required electric power;
thereby during a medical checkup, said setting unit is used to set said standard value again according actual conditions including the recent health measuring standard announced by WHO or ADA or the instructions given by doctors, and said values are stored into said data storage unit, so that during a medical checkup, said recently set standard values and said measured values are compared, and the result is displayed by said display unit and reminded by said reminding unit.

2. The biosensor capable of setting standard measuring values of claim 1, wherein said setting unit is a press button.

3. The biosensor capable of setting standard measuring values of claim 1, wherein said display unit is a liquid crystal display module.

4. The biosensor capable of setting standard measuring values of claim 1, wherein said data storage unit is an external memory.

5. The biosensor capable of setting standard measuring values, wherein said processor is coupled to a reminding unit.

6. The biosensor capable of setting standard measuring values of claim 5, wherein said reminding unit is a light signal.

7. The biosensor capable of setting standard measuring values of claim 5, wherein said reminding unit is a loudspeaker.

8. The biosensor capable of setting standard measuring values of claim 1, wherein said data storage unit includes a body mass index (BMI) setting mode, so that a measurer can use said setting unit during a medical checkup to input the values required by said body mass index (BMI), and said processor stores said values in said data storage unit.
